(19) United States
(12) Patent Application Publication
(10) Pub. No.: US 2014/0066895 A1
(43) Pub. Date: Mar. 6, 2014

(54) ANATOMIC DEVICE DELIVERY AND POSITIONING SYSTEM AND METHOD OF USE

(71) Applicant: Robert KIPPERMAN, Chatham, NJ (US)

(72) Inventor: Robert KIPPERMAN, Chatham, NJ (US)

(21) Appl. No.: 13/835,650

(22) Filed: Mar. 15, 2013

Related U.S. Application Data
(60) Provisional application No. 61/694,486, filed on Aug. 29, 2012.
(60) Provisional application No. 61/700,096, filed on Sep. 12, 2012.

(51) Int. Cl. A61M 25/09

(52) U.S. Cl. CPC A61M 25/09
USPC 604/509; 604/104

(57) ABSTRACT

An anatomic device delivery and positioning system has a stabilizing guide wire for placement of a catheter or other medical device or material into a vessel or cavity of a tissue or organ within a body of a living subject into which the guide wire is insertable. The guide wire includes an elongated member having a proximal end and a distal end, the proximal end to extend out of the body and the distal end to extend into the body, the distal end having an expandable portion that expands from a compressed condition when inside of a delivery tube or catheter to an expanded condition when outside of the delivery tube or catheter. A method is also disclosed of performing a percutaneous procedure within a vessel or cavity of a tissue or organ of a subject using the guide wire, particularly but not exclusively involving a heart procedure.

### CROSS-REFERENCE TO RELATED APPLICATIONS

This application claims the benefit under U.S. Provisional Patent Application 61/694,486, filed Aug. 29, 2012, and U.S. Provisional Patent Application 61/700,096, filed Sep. 12, 2012, the disclosures of which are hereby incorporated by reference herein, in their entireties.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

1. Field of the Invention

The present invention relates to an anatomic device delivery and positioning system having a stabilizing guide wire that is used to place a catheter or other medical device within a vessel or cavity of a tissue or organ within a subject that may be an animal or human. More particularly, the invention relates to an anatomic device delivery and positioning system comprising a guide wire having a distal end having a specialized shape for delivering a medical device where the distal end is self-centering within a subject's vessel or cavity of a tissue or organ and non-injurious to the subject. The distal end is compressible to fit in a delivery component and adapted for placement within living anatomy to stabilize and allow more precise placement of a medical device, such as a diagnostic or interventional catheter and associated medical equipment, treatment or repair devices and materials, within a vessel or cavity of an organ, preferably within a human and more preferably within a human heart.

2. Background Information

In the field of medicine, especially vascular medicine, and more specifically cardiology, various catheters are placed in vessels and cardiac chambers. More broadly in the vascular space catheters that are used to deliver specialized medical tools, treatment products and materials, ranging from joint components to replacement heart valves and myriad others of a scope and complexity that seemingly increases daily, are usually placed over a guide wire that had been inserted into a vessel, tissue or organ in a manner to prevent damage to the vessel, tissue or organ. The present invention has applications in a variety of interventional and transapical applications, as well as applications involving a cut down. It can involve potentially any vessel, tissue or organ in a human or non-human animal body, but is especially desirable for treating humans. It may be used for, among other indications, cardiac interventions, kidney-related treatments such as and renal denervation, and neurological diagnostic and treatment indications, as well as treatment of conditions and indications of upper and lower extremities, particularly those relating to vascular indications. While a great many examples could be presented where the delivery system of the present invention would be useful, to help direct attention to perhaps the most likely indications, this disclosure will focus, in an exemplary and non-limiting manner, on cardiological indications. For example, in the case of percutaneous aortic valve replacement, the catheter is advanced to the aortic position over a guide wire which has been placed in the left ventricle. Currently, these guide wires are very stiff to support large bore catheters. Usually the guide wires have a floppy distal end that prevents damage to the ventricle. Typically the distal end is manually shaped in a manner to make it less traumatic to the ventricle, but still it can sometimes cause trauma. Other exemplary cardiological procedures include right heart pacing (especially on an emergency basis), placement of left atrial appendage devices, and placement of percutaneous valve repair systems.

In many current delivery systems involving catheterization and guide wires, the insertion of the guide wire does not provide any mechanism for actively determining the course of the catheter. The guide wire conforms to the anatomy of the chamber in which it resides and determines the course of the catheter. Accurate placement of medical devices and materials, for instance cardiac devices, such as replacement valves, depends on the course of the guide wires, which do not move in a precise way and the course of inserting the guide wires do not have a predictable manner of movement when being inserted.

It is especially important in various medical procedures, and particularly cardiac procedures, such the transcatheter aortic valve implementation (TAVI) procedure, to have a predictable and controllable insertion of the guide wire to appropriately position the catheter and any implements, replacement valves, etc., delivered through the catheter to perform the procedure. In this procedure a stiff guide wire (usually an Amplatz® Super Stiff™ guide wire) has its distal end shaped into a curve that approximates the shape of the left ventricular apex. The guide wire is usually placed in the left ventricle apex with a pigtail catheter. The guide wire is positioned so that the elbow of the curve is in the apex, thus allowing the ability for the surgeon to push on the guide wire with decreased risk of perforation. This guide wire is used as a rail for large catheters in percutaneous aortic valvuloplasty (PAV). As the catheter is advanced over the guide wire, forward forces are transferred to the distal end of the guide wire. The guide wire determines the course of the catheter and the anatomy determines the course of the guide wire in a passive manner. Accurate placement of the catheter is difficult as the guide wire is not moveable or manipulatable in a predictable way and is not able to pivot.

There is clearly a need to have an improved anatomic device delivery and positioning system particularly using a guide wire that allows good support for catheter insertion with less chance of perforation of vessels, tissues or organs into or through which the guide wire is inserted, and a need for some flexibility for spatial orientation of the guide wire during and upon completion of insertion into the desired vessel, tissue or organ. The present invention satisfies that need.

The anatomic device delivery and positioning system comprises a stabilizing guide wire of the present invention is a stiff wire with a distal end that is expandable to a three dimensional shape, thus allowing pivoting in three dimensions and allowing transition of the force to a larger area. This stabilizing guide wire can adapt to the shape of the left ventricle apex or other vessel, tissue or organ structural shapes and yet still can be collapsible into a delivery device such as a tube or catheter, for instance an exchange catheter, a sheath or a tube within a tube type of coaxial guide wire system facilitating the use of large bore catheters for various procedures, many involving cardiology, as well as the use of much smaller catheters such as those used for neurological interventions. The present invention overcomes the problems of the prior devices and is well-suited for an efficient performance of various transapical and percutaneous procedures involving catheter access to various organs, particularly heart procedures, while minimizing the risks inherent in such procedures. Such procedures include without limitation transcatheter aortic valve implementation (TAVI), transcatheter pulmonic valve implementation (TPVI), transcatheter tricuspid valve implementation, percutaneous mitral valve implementation, percutaneous mitral valve repair, transcatheter tricuspid valve repair, and mitral pulmonic and aortic valvuloplasty, among other procedures, as well as right heart pacing (especially on an emergency basis), placement of left atrial appendage devices, and placement of other percutaneous valve repair systems.

### DEFINITIONS

As used herein, the singular forms “a”, “an”, and “the” include plural referents, and plural forms include the singular referent unless the context clearly dictates otherwise.

As used herein, the term “about” with respect to any numerical value, means that the numerical value has some reasonable leeway and is not critical to the function or operation of the component or portion of the guide wire or its components or tubes or catheters with which they are used that are being described or the method with which the guide wire is used, and will include values within plus or minus 5% of the stated value.

As used herein, the term “generally” or derivatives thereof with respect to any element, portion or parameter, means that the element, portion or parameter has the basic shape, or the parameter has the same basic direction, orientation or the like to the extent that the function of the element, portion or parameter would not be materially adversely affected by somewhat of a change in the element, portion or parameter, such as an effort to design around the element, portion or parameter, while maintaining its essential form, shape or function. By way of example and not limitation, a shape of the expandable portion of the guide wire described as “generally ovoid” or “generally spherical” need not be absolutely ovoid or spherical, and structural elements referred to as “generally longitudinal” or “generally transverse” need not be respectively absolutely longitudinal with reference to a longitudinal axis or perpendicular with reference to the longitudinal axis of the guide wire.

### BRIEF SUMMARY OF THE INVENTION

One aspect of the present invention relates to an anatomic device delivery and positioning system having a guide wire for guiding placement of a delivery device, such as a catheter, into a vessel or cavity of a tissue or organ within a body of a living subject into which the guide wire is insertable, wherein the guide wire comprises an elongated member having a proximal end and a distal end, the proximal end to extend out of the body and the distal end to extend into the body, the distal end having an expandable portion that expands from a compressed condition when inside of a delivery device such as a tube or catheter to an expanded condition when outside of the delivery tube or catheter.

Another aspect of the invention relates to methods of using the anatomic device delivery and positioning system including a stabilizing guide wire of the present invention in various interventional procedures, such as apical and percutaneous procedures, involving a human or animal subject. Accordingly, this aspect relates to a method of performing a procedure comprising inserting the guide wire used with the present invention into a delivery structure like a transfer tube, guiding the delivery structure transfer tube containing the guide wire including its expandable portion in a compressed condition through a delivery tube into an approximate position within a vessel or cavity of a tissue or organ of a subject, retracting the delivery structure transfer tube, extending the guide wire from the delivery tube such that the expanded portion of the guide wire expands to its expanded condition, and positioning the expanded portion of the guide wire in its expanded position to a final desired location within the vessel or cavity of a tissue or organ of the subject. Particularly preferred are methods involving surgical procedures within the human heart.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is a side elevation or top plan view of one embodiment of a stabilizing guide wire according to the present invention, where an intermediate portion of the guide wire is truncated, showing the expandable portion of a first exemplary generally ovoid shape at the distal end in an expanded condition;

FIG. 2 is a side elevation or top plan view of another embodiment of a stabilizing guide wire according to the present invention, where an intermediate portion of the guide wire is truncated, showing the expandable portion of a second exemplary generally spherical shape at the distal end in an expanded condition;

FIG. 3 is a side elevation or top plan view of yet another embodiment of a stabilizing guide wire according to the present invention, where an intermediate portion of the guide wire is truncated, showing the expandable portion of an exemplary generally spherical shape with more structural elements, including generally longitudinal and generally transverse wire elements, at the distal end in an expanded condition;

FIG. 4 is a side elevation or top plan view of the embodiment of the stabilizing guide wire as shown in FIG. 3, where an intermediate portion of the guide wire is truncated, showing the expandable portion at the distal end in an expanded condition and a transfer tube, truncated at its proximal portion, inserted over the proximal end of the guide wire;

FIG. 5 is a side elevation or top plan view of the embodiment of the stabilizing guide wire as shown in FIG. 4, where the transfer tube, truncated at its proximal portion, is advanced along the guide wire toward the distal end of the guide wire;

FIG. 6 is a side elevation or top plan view of the embodiment of the stabilizing guide wire as shown in FIG. 5, where the expandable portion at the distal end of the guide wire is beginning to be compressed into its compressed condition as it enters the distal end of the transfer tube, truncated at its proximal portion;

FIG. 7 is a side elevation or top plan view of the embodiment of the stabilizing guide wire as shown in FIG. 6, where the expandable portion at the distal end of the guide wire is compressed into its compressed condition within the distal end of the transfer tube, truncated at its proximal portion;

FIG. 8 is a side elevation or top plan view of the embodiment of the stabilizing guide wire as shown in FIG. 7, where the expandable portion at the distal end of the guide wire is compressed into its compressed condition within the distal end of the transfer tube, truncated at its proximal portion, and where the transfer tube with the compressed expandable portion of the guide wire is shown coaxially entering the proximal end of a delivery tube, the distal end of the delivery tube being truncated;

FIG. 9 is a side elevation or top plan view of the embodiment of the stabilizing guide wire as shown in FIG. 8, where the expandable portion at the distal end of the guide wire is compressed into its compressed condition within the delivery tube after the transfer tube has been removed from the delivery tube and the guide wire, where the delivery tube has been advanced along the guide wire toward the distal ends of the delivery tube and the guide wire, an intermediate portion of the delivery tube being truncated;

FIG. 10 is a side elevation or top plan view of the embodiment of the stabilizing guide wire as shown in FIG. 9, where the expandable portion at the distal end of the guide wire is expanded into its expanded condition upon exiting from the distal end of the delivery tube, an intermediate portion of the delivery tube being truncated;

FIG. 11 is a side elevation view of another embodiment of the stabilizing guide wire, similar to the embodiment of FIG. 3, where the expandable portion at the distal end of the guide wire is expanded into its expanded condition, and where the elongated member of the guide wire is a tube, and further showing an optional unipolar pacing electrode used in some procedures at the expandable portion of the guide wire in the expanded condition, where a wire connected to the unipolar pacing electrode extends through and out the proximal end of the elongated member tube, intermediate portions of the tube and pacing electrode wire being truncated;

FIG. 12 is a side elevation view of still another embodiment of the stabilizing guide wire, similar to the embodiment of FIG. 3, where the expandable portion at the distal end of the guide wire is expanded into its expanded condition, and where the elongated member of the guide wire is a tube, and further showing optional bipolar pacing electrodes used in some procedures at the expandable portion of the guide wire in the expanded condition, where wires connected to the bipolar pacing electrodes extend through and out the proximal end of the elongated member tube, intermediate portions of the tube and pacing electrode wires being truncated;

FIG. 13 is a schematic representation, in a side elevation view, partly in cross-section, showing the use of a prior art guide wire having a “J-curve” distal end in a transcatheter aortic valve implementation method where the J-curve portion is in the left ventricle in a living heart shown schematically in cross-section within a subject's body; and

FIG. 14 is a schematic representation, similar to FIG. 13, in a side elevation view, showing the use of the embodiment of the stabilizing guide wire of the present invention shown in FIG. 3 in a transcatheter aortic valve implementation method according to the present invention where the expandable portion at the distal end of the guide wire is shown seated at the apex of the left ventricle in a living heart shown schematically in cross-section within a subject's body.

### DETAILED DESCRIPTION OF THE INVENTION

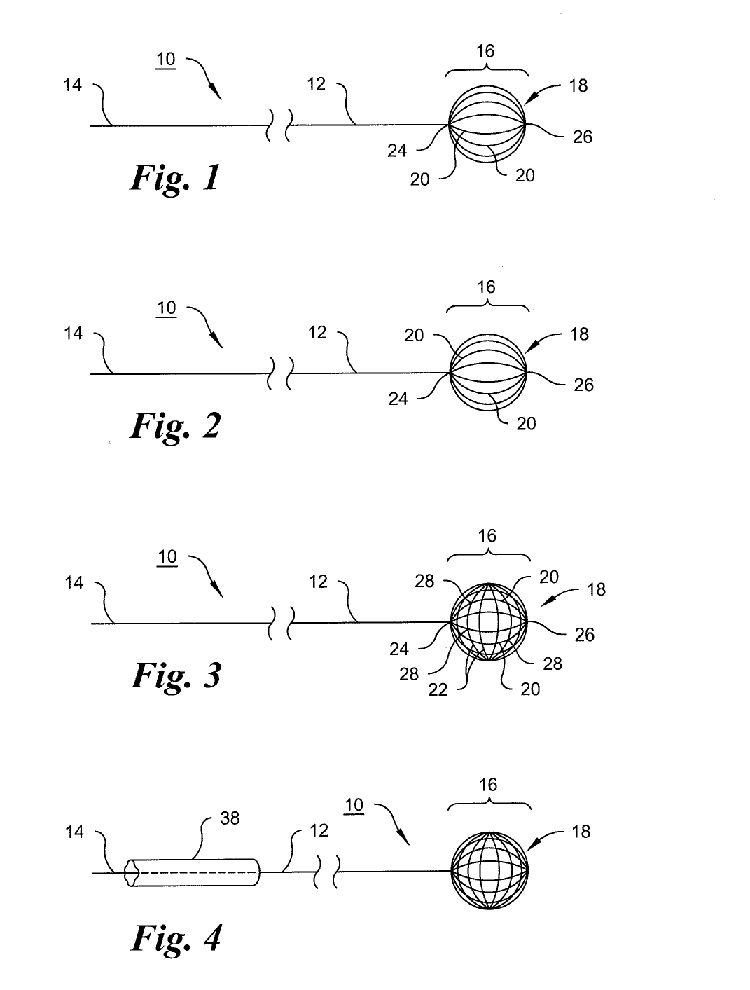

With reference to the drawings, where like numerals refer to like elements throughout the several views, there are shown in FIGS. 1-3 three embodiments of an anatomic device delivery and positioning system according to the present invention having a stabilizing guide wire 10 comprising an elongated member 12, where an intermediate portion of the guide wire is shown truncated in the figures due to its length. The elongated member 12 of the guide wire 10 has a proximal end 14 and a distal end 16. Three different embodiments of an expandable portion 18 located at the distal end 16 in its expanded condition are shown in FIGS. 1-3. In FIG. 1, the expandable portion 18 in its expanded condition is shown as having a generally ovoid shape. In FIG. 2, the expandable portion 18 in its expanded condition is shown as having a generally spherical shape. In FIG. 3, the expandable portion 18 in its expanded condition is shown as having a generally spherical shape, but comprising generally longitudinal structural elements 20, and generally transverse wires 22, whereas in the embodiments of FIGS. 1 and 2, the expandable portion is shown with only generally longitudinal structural elements 20. The generally spherical shape is a preferred shape for the expandable portion 18 in its expanded condition, but other three dimensional shapes for the expandable portion when in the expanded condition are feasible, for example without limitation, semi-spheroidal, three-sided or four-sided pyramidal, tetrahedral or octahedral, with all potentially relatively sharp edges and tips being blunted or truncated to prevent or significantly lessen the chance that a perforation or other trauma or damage to any vessel or cavity of a tissue or organ will occur. Details of the expandable portion 18 will be set forth hereinafter.

The wire material used for the guide wire 10 up to the expandable portion 18 at the distal end 16 can be any material of suitable stiffness, such as, without limitation, the wire used for the Amplatz® Super Stiff™ guide wires and the Lunderquist™ guide wires. In general, the guide wire 10 should have a stiffness suitable for positive placement within a vessel, tissue or organ or within a cavity in such a tissue or organ while being flexible enough to follow the vessels of human or animal anatomy. The placement and orientation of the guide wire 10 is aided by the usual x-ray, fluoroscopic, ultrasonic, echo such as transesophageal echocariography, magnetic resonance, optical coherence tomography or other tomographic imaging techniques, among any other suitable medical imaging, based on at least one image-opaque marking near and on the distal end 16, including the distal end of the expanded portion 18.

The stiffness of the guide wire 10 is measured by determining its flexural modulus. For methods of determining the stiffness of guide wires, reference is made to G. J. Harrison, et al., “Guidewire Stiffness: What's in a Name?” J. Endovasc. Ther., 2011: 18:797-801 (“Harrison”), the disclosure of which is hereby incorporated herein in its entirety. Using the Harrison method of determining stiffness of guide wires, the guide wire 10 of the present invention may typically have a stiffness of about 8 gigapascals [GPa] (about 8,000 newtons/square millimeter [N/mm²] or about 1,160,302 pounds per square inch [psi]) to about 200 GPa (about 200,000 N/mm² or about 29,007,548 psi); preferably about 16 GPa (about 16,000 N/mm² or about 2,320,804 psi) to about 170 GPa (about 170,000 N/mm² or about 24,656,415 psi); and more preferably, about 55 GPa (about 55,000 N/mm² or about 7,977,076 psi) to about 160 GPa (about 160,000 N/mm² or about 23,206,038 psi).

The guide wire 10 may be of any length that is convenient for a given procedure, within a broad range of about 15 cm (5.9 inch) to about 300 cm (118.1 inches). The guide wire 10 may be supplied in any desired length, including non-limiting exemplary lengths for guide wires of 100 cm (39.4 inches) and 260 cm (102.4 inches). A long guide wire 10 may be cut at its proximal end 14 if there is a need to shorten it.

The elongated member 12 of the guide wire 10 from the proximal end 12 to the distal end 14 just short of the expandable portion 18 may be made of any suitable and usual wire material used to make guide wires having the required stiffness and flexibility as discussed above. Suitable materials include, without limitation, titanium, vanadium, aluminum, nickel, iron, tantalum, zirconium, chromium, silver, gold, silicon, magnesium, niobium, scandium, platinum, cobalt, palladium, manganese, molybdenum, and alloys of any two or more thereof. Stainless steel and nitinol are preferred materials for this portion of the guide wire 10.

The elongated member 12 may be at least partially or fully coated with any usual material for easing the guide wire 10 though body vessels, or treating the vessel, tissue or organ into which the guide wire travels. The coating may be a synthetic polymer, for instance silicone, polytetrafluoroethylene or polyurethane. Various other well-known hydrophilic, hydrophobic or other coatings may also be applied so long as the coatings do not adversely affect the stiffness and flexibility requirements of the guide wire 10.

The guide wire 10 must have a diameter to fit within the lumens of appropriate delivery devices, typically and without limitation transfer tubes, delivery tubes and catheters, potentially along with other adjunct equipment, such as optional pacing electrodes and electrode wires, for example, as well as materials to be delivered and accurately positioned and placed within a vessel, or organ or tissue cavity. Transfer tubes and some delivery tubes are usually measured in “Gauge” units, where the larger the gauge the smaller the diameter, and have various wall thicknesses from regular to thin to extra thin walls, varying greatly in outer and inner diameters. For example, one manufacturer supplies transfer tubes and delivery tubes as stainless steel 304 hypodermic tubing with many sizes ranging from 33 Gauge having an outer diameter of 0.008 inch (0.20 mm) and an inner diameter of 0.0035 inch (0.09 mm) up to 6 Gauge having an outer diameter of 0.2 inch (5.08 mm) and an inner diameter of 0.17 inch (4.32 mm). Delivery tubes and catheters used in various transcutaneous procedures, such as those used relating to the human heart, typically have an outer diameter, measured in “French,” of about 4 French to about 12 French, where the larger the French unit the larger the diameter. One French unit equals 0.33 mm (0.013 inch). Delivery tubes and catheters of these outer diameters have varying inner diameters, depending on the material of which the delivery tubes and catheters are made. In any event, the guide wire 10, including the expandable portion 18 in its compressed condition, must be able to fit through lumens of a given inner diameter.

The elongated member 12 of the guide wire 10 generally should have a diameter of about 0.009 inch (0.23 mm) to about 0.064 inch (1.63 mm), preferably about 0.013 inch (0.33 mm) to about 0.050 inch (1.27 mm), and more preferably about 0.035 inch (0.89 mm). The diameter of the elongated member 12 can be selected based on the type of the procedure for which the delivery and positioning system is used. For example, neurological interventions may use guide wires with smaller diameters, on the order of about 0.014 inch (0.356 mm) that may fit within smaller tubular delivery devices having an inner diameter of about 0.016 inch (0.406 mm), while other procedures such as cardiac interventions, may use guide wires with stiffer diameters that can be accommodated along with other materials, such as replacement heart valves, within delivery devices such as sheaths and catheters having a larger inner diameter.

As described so far, the elongated member 12 of the guide wire 10 of the present invention is conventional in shape, size, stiffness and materials as prior art guide wires, such as the Amplatz® Super Stiff™ guide wires and the Lunderquist™ guide wires, which have distal tips of various shapes and radii of curvature. The unique and inventive aspect of the guide wire 10 of the present invention is the expandable portion 18 that may be attached to the distal end of the elongated member 12 by spot welding, soldering or adhesive bonding with medical grade epoxies or other adhesives, for example. If the elongated member 12 is made of nitinol or other material of which the expandable portion 18 is also made, such as strands of braided or bonded nitinol wire, then the expandable portion 18 may be unitary and integral with the elongated member 12, rather than the expandable portion 18 being separately attached and therefore integral with the elongated member 12.

As shown in FIGS. 1-3, the expandable portion 18, in its expanded condition, may have various shapes and configurations. All of such shapes and configurations have as common features a greater surface area than any known guide wire having any distal end shape or radius of curvature. The expanded portion 18 in its expanded condition bears gently but firmly against the walls of a cavity of a vessel, tissue or organ into which it is inserted to allows the physician or other health professional to manipulate and maneuver the guide wire such that both the elongated member 12 and the distal end 16 are positioned in just the right locations leading to and at the final determined resting location depending on the procedure being performed. This assures appropriate placement of the catheter and any ancillary equipment, replacement heart valves, or the like delivered via the catheter or other delivery devices to the location of the procedure in the cavity of the vessel, tissue or organ. The guide wire 10, with its expandable portion 18 provides the physician with the ability to push on the guide wire without injury to the vessel, tissue or organ in which the expandable portion 18 is located and expanded, and to manipulate and maneuver the guide wire 10 into its desired position. The expandable portion 18 in its expanded condition acts as an anchor that allows self centering of the guide wire in a cavity, such as, without limitation, along the center of the long axis of a ventricle, like the left ventricle in a human heart. In addition, it allows pivoting of the guide wire, allowing it to move in multiple angles and directions easily, thus aiding in its proper positioning at its ultimate destination within a cavity of a vessel, tissue or organ.

As mentioned above, the expandable portion 18 may have various three dimensional shapes, so long as it is compressible within a delivery device and when it is in its expanded condition, it is conformable to the desired ultimate location, based on the procedure involved. The expandable portion 18 is presently preferred to be generally ovoid-shaped or generally spherical-shaped.

The material of the expandable portion 18 has a structure that is compressible within or by a delivery device such as a delivery or transfer tube, sheath or catheter and is expandable and conformable, preferably in advance, to the ultimate desired location. The expandable portion 18 may be made of a conformable synthetic polymeric material, such as polyurethane that may be formed, such as by various molding techniques, to a desired final expandable form. The expandable portion may and preferably does have shape memory capability that may or may not be temperature activated.

Alternatively, the expandable portion 18 may be a fluid-expandable balloon, where the expanding fluid may be delivered to the balloon by a separate tube coaxial with or adjacent to the guide wire elongated portion 12. Ideally, for this embodiment, the elongated portion could be tubular, such that the expanding fluid could be delivered to the balloon through the tube's lumen.

The expandable portion 18 also may be made and as presently preferred would be made using a metal selected from the group consisting of titanium, vanadium, aluminum, nickel, iron, tantalum, zirconium, chromium, silver, gold, silicon, magnesium, niobium, scandium, platinum, cobalt, palladium, manganese, molybdenum, and alloys of any two or more thereof. Preferably, the material is of nitinol and stainless steel, and more preferably nitinol.

Nitinol is a well-known alloy of nickel and titanium, in roughly equal atomic percentages that has excellent biocompatibility with human and animal tissues. Nitinol alloys exhibit two closely related and unique properties: shape memory and superelasticity (also called pseudoelasticity). Shape memory refers to the ability of a material to undergo deformation at one temperature, then recover its original, non-deformed shape upon heating above its “transformation temperature.” Superelasticity occurs at a narrow temperature range just above its transformation temperature; but in this case, no heating is necessary to cause the non-deformed shape to recover. Nitinol exhibits enormous elasticity, on the order of about 10 to 30 times that of ordinary metal. Austenite is nitinol's stronger, higher temperature phase, where its crystalline structure is simple cubic. Superelastic behavior is in the phase (over a 50° C.-60° C. temperature spread). Martensite is nitinol's weaker, lower temperature phase, where its crystalline structure is twinned and is easily deformed in this phase. Once deformed in the martensite phase, it will remain deformed until heated to its austenite phase, where it will return to its pre-deformed shape, with shape memory effect.

The expandable portion 18 thus may be made in its expanded condition to be in the higher temperature found in living animals and humans, such that the expanded condition is its normal condition, unless subjected to lower temperatures or mechanical stress as applied when the expandable portion 18 is inserted into a transfer tube or delivery tube or catheter.

The expanded portion 18, when made of a structured material, rather than from a preformed, such as moldable material, may be made of interconnected structural elements of nitinol or other material that are arranged generally longitudinally as schematically shown in FIGS. 1 and 2, respectively showing embodiments of the generally ovoid-shaped or generally spherical-shaped expandable portion 18. In these embodiments, the generally longitudinal structural elements are bonded where they are joined together at the proximal end 24 of the expandable portion where the expandable portion 18 joins with the distal end of the elongated member 12, and at the distal end 26 of the expandable portion 18, which is the very distal end of the overall guide wire. As schematically shown in FIG. 3, the expandable portion 18 has an exemplary spherical shape with more structural elements, including generally longitudinal structural elements 20 and generally transverse structural elements 24, which may be bonded at least at some of their intersecting points 28, forming generally rectangular spaces between the intersecting points 28 in the expanded condition. In the compressed condition or as the expandable portion 18 is being compressed, the generally rectangular spaces become flattened to generally diamond-shaped spaces. Ultimately the structural elements 20 and 22 contact each other when in the fully compressed condition.

The structural elements 20 and 22 of the expandable portion 18 may be wires spot welded or braided as schematically illustrated in the drawings to form the overall expandable portion 18. Alternatively, the structural elements may be formed by other well-known techniques, such as molding, electrodeposition, vacuum deposition, laser etching, chemical etching, or the like, where the interconnections among the structural elements are created by the pattern used when the expandable portion 18 is formed.

In addition to nitinol, other similar shape-retention materials that may be used for the expandable portion include, without limitation, cobalt-based low thermal expansion alloy referred to in the field as ELGELOY®, nickel-based high temperature high-strength superalloys commercially available from Haynes International, Inc. under the trade name HASTELLOY®, nickel-based heat treatable alloys sold under the name INCOLOY® by Special Metals Corporation group of companies, a part of Precision Castparts Corp., and a number of different grades of stainless steel. The important factor in choosing a suitable material for the material is that the wires or other formed structural elements retain a suitable amount of the deformation induced by a molding surface or as otherwise formed when subjected to a predetermined heat treatment.

U.S. Pat. Nos. 4,991,602, 5,067,489, 5,846,251 and 6,695,865 disclose various methods and techniques for using shape memory alloys, and particularly nitinol, in guide wires and other medical products relating to percutaneous procedures. The disclosure of these patents are hereby incorporated herein by reference in their entireties.

For example, the expandable portion 18 could be formed as described in U.S. Pat. No. 5,846,251, as follows. An appropriately sized piece of tubular or planar metal fabric is inserted into a mold, whereby the fabric deforms to generally conform to the shape of the cavities within the mold. The shape of the cavities is such that the metal fabric deforms into substantially the shape of the desired expandable portion 18, such as generally ovoid or generally spherical. The ends of the wire strands of the tubular or planar metal fabric forming the structural elements 20 and 22 should be secured to prevent the metal fabric from unraveling. A clamp or welding, as further described below, may be used to secure the ends of the wire strands.

In the case of a tubular braid, a molding element may be positioned within the lumen of the braid prior to insertion into the mold to thereby further define the molding surface. If the ends of the tubular metal fabric have already been fixed by a clamp or welding, the molding element may be inserted into the lumen by manually moving the wire strands of the fabric apart and inserting the molding element into the lumen of the tubular fabric. By using such a molding element, the dimensions and shape of the finished medical device can be fairly accurately controlled and ensures that the fabric conforms to the mold cavity.

The molding element may be formed of a material selected to allow the molding element to be destroyed or removed from the interior of the metal fabric. For example, the molding element may be formed of a brittle or friable material. Once the material has been heat treated in contact with the mold cavities and molding element, the molding element can be broken into smaller pieces which can be readily removed from within the metal fabric. If this material is glass, for example, the molding element and the metal fabric can be struck against a hard surface, causing the glass to shatter. The glass shards can then be removed from the enclosure of the metal fabric.

Alternatively, the molding element can be formed of a material that can be chemically dissolved, or otherwise broken down, by a chemical agent which will not substantially adversely affect the properties of the material used for the expandable portion 18. For example, the molding element can be formed of a temperature resistant plastic resin which is capable of being dissolved with a suitable organic solvent. In this instance, the fabric and the molding element can be subjected to a heat treatment to substantially set the shape of the fabric in conformance with the mold cavity and molding element, whereupon the molding element and the metal fabric can be immersed in the solvent. Once the molding element is substantially dissolved, the metal fabric can be removed from the solvent.

Care should be taken to ensure that the materials selected to form the molding element is capable of withstanding the heat treatment without losing its shape, at least until the shape of the fabric has been set. For example, the molding element could be formed of a material having a melting point above the temperature necessary to set the shape of the wire strands, but below the melting point of the metal forming the strands. The molding element and metal fabric can then be heat treated to set the shape of the metal fabric, whereupon the temperature can be increased to substantially completely melt the molding element, thereby removing the molding element from within the metal fabric.

When the tubular braid for example is in its relaxed configuration, the wire strands forming the tubular braid will have a first predetermined relative orientation with respect to one another. As the tubular braid is compressed along its axis, the fabric will tend to flare out away from the axis conforming to the shape of the mold. When the fabric is so deformed the relative orientation of the wire strands of the metal fabric will change. When the mold is assembled, the metal fabric will generally conform to the molding surface of the cavity. The expandable portion 18 has a preset expanded condition and a compressed condition which allows the guide wire 10 with its expandable portion 18 in its compressed condition to be passed through a delivery tube, which may be a stainless steel or other metal tube or catheter made of the typical synthetic polymers or other similar transfer device. The expanded configuration is generally defined by the shape of the fabric when it is deformed to generally to conform to the molding surface of the mold.

Once the tubular or planar metal fabric is properly positioned within a preselected mold with the metal fabric generally conforming to the molding surface of the cavities therein, the fabric can be subjected to a heat treatment while it remains in contact with the molding surface. Heat treating the metal fabric substantially sets the shapes of the wire strands in a reoriented relative position when the fabric conforms to the molding surface. When the metal fabric is removed from the mold, the fabric maintains the shape of the molding surfaces of the mold cavities to thereby define a medical device having a desired shape. This heat treatment will depend in large part upon the material of which the wire strands of the metal fabric are formed, but the time and temperature of the heat treatment should be selected to substantially set the fabric in its deformed state, i.e., wherein the wire strands are in their reoriented relative configuration and the fabric generally conforms to the molding surface.

After the heat treatment, the fabric is removed from contact with the molding element and will substantially retain its shape in a deformed state. If a molding element is used, this molding element can be removed as described above.

The time and temperature of the heat treatment can very greatly depending upon the material used in forming the wire strands. As noted above, one preferred class of materials for forming the wire strands are shape memory alloys, with nitinol being particularly preferred.

As also described above, the expandable portion 18 may be made of a conformable synthetic polymeric material, such as polyurethane that may be formed, such as by various molding techniques, to a desired final expandable form. The expandable portion may and preferably does have shape memory capability that may or may not be temperature activated.

The expandable portion 18, when in the expanded condition, may have a maximum transverse cross-sectional dimension, such as when in a generally ovoid shape or a diameter when in a generally spherical shape (the maximum transverse cross-sectional distance or diameter of the expandable portion of any given shape being referred to herein merely as “diameter” for the sake of convenience), of about 0.02 inch (0.5 mm) to about 1.57 inches (4 cm), preferably about 0.59 inch (1.5 cm) to about 1.18 inches (3 cm), and more preferably about 0.78 inch (2 cm).

The expandable portion 18, when in the compressed condition, must have a maximum transverse cross-sectional dimension (as above, “diameter” for the sake of convenience) that will allow the guide wire 10, including the expandable portion 18 in its compressed condition to pass through the lumen of a delivery device such as a transfer tube, delivery tube or catheter, with or without an overlying sheath, having various predetermined lumen inner diameters as discussed above. Preferably, the expandable portion 18, when in the compressed condition, has a diameter of about 0.01 inch (0.25 mm) to about 0.082 inch (2.3 mm), and more preferably about 0.056 inch (1.42 mm) to about 0.070 inch (1.78 mm).

As mentioned above, there are some procedures, such as neurological interventions, where the smallest possible dimensions are desirable for the guide wire 10 and its elements, including the elongated portion 12 and the expandable portion 18, consistent with good delivery and positioning in the ultimate location. Accordingly, all of the dimensions set forth herein are exemplary, rather than limiting.

In some heart procedures, temporary cardiac pacing is needed, and in some situations, such as emergencies, temporary pacing can be lifesaving. It involves electrical cardiac stimulation to treat a tachyarrhythmia or bradyarrhythmia until it resolves or until long-term therapy can be initiated. The purpose of temporary pacing is the reestablishment of circulatory integrity and normal hemodynamics that are acutely compromised by a slow or fast heart rate by maintaining an appropriate heart rate. Transvenous cardiac pacing, also called endocardial pacing, is one type of an intervention that could be performed with the device of the present invention, and that can be used to treat symptomatic arrythmias that do not respond to transcutaneous pacing or to drug therapy. Transvenous pacing is achieved by threading a pacing electrode or pair of electrodes through a vein into the right atrium, right ventricle, or both. The pacing wire or wires are then connected to an external pacemaker outside the body. Transvenous pacing is often used as a bridge to permanent pacemaker placement. It can be kept in place until a permanent pacemaker is implanted or until there is no longer a need for a pacemaker and then it is removed. During TAVI procedures, rapid ventricular pacing is often performed in order to lower the patient's blood pressure to assure more accurate placement of the aortic valve being implanted. Endocardial pacing from the left ventricle with a guide wire such as guide wire 10 as shown in the optional, alternative embodiments of FIGS. 11 and 12 obviates the need for a separate temporary transvenous pacing procedure.

FIGS. 11 and 12 show optional, alternative embodiments of the guide wire 10 of the present invention for use where venous cardiac pacing is indicated. These embodiments include, in FIGS. 11 and 12 respectively, a unipolar pacing electrode 30 and bipolar pacing electrodes 32 that are connected to the structure elements 20 and 22 forming the expandable portion 18. The electrodes are capable of contacting the tissue of the tissue or organ cavity into which it is insertable. In these embodiments, the elongated member 12 is tubular having the same properties of stiffness, flexibility and ability to manipulate and maneuver the expandable portion 18 through and into vessels, tissues and organs and their cavities as the embodiments shown in the other drawings and as described above regarding the elongated member 12 being a wire. The tube of the elongated member may be a hypodermic tube of stainless steel, nitinol or other material used to make the elongated member 12 of wire, with a suitable inner diameter to retain an electrode lead wire 34 connected to the unipolar pacing electrode 30 and a pair of lead wires 36 connected to the bipolar pacing electrodes 32, where the wires extend out of the proximal end 14 of the tubular elongated member 12. The tubular elongated member 12 has an outer diameter that is as small as possible to provide the tube with structural integrity yet sill fit within a transfer tube and delivery tube or catheter as described above regarding the embodiment where the elongated member 12 is a wire, rather than a tube.

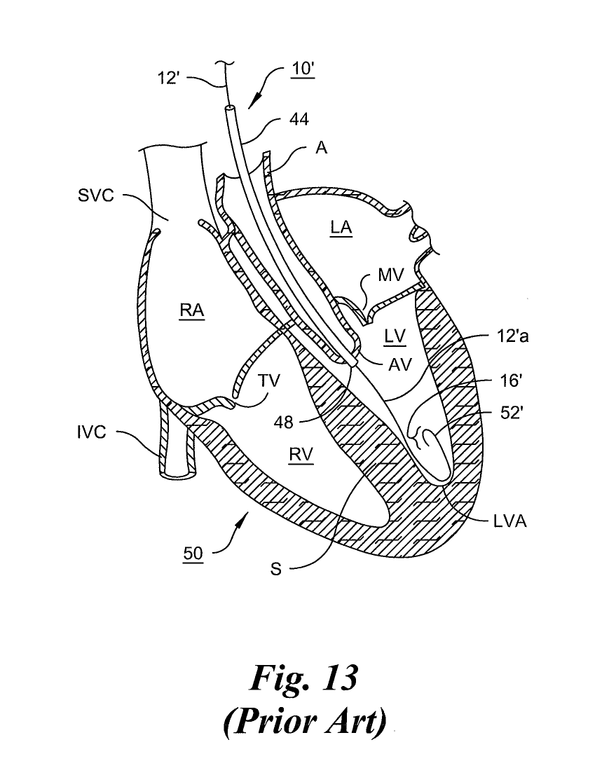

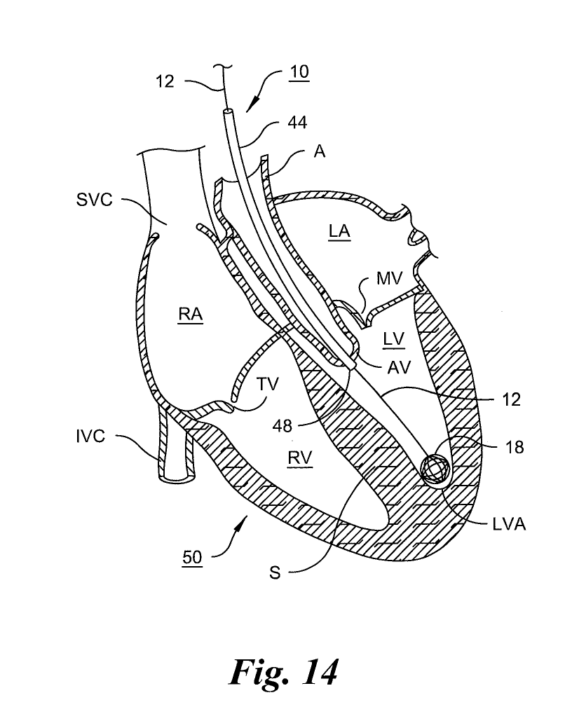

The use of the anatomic device delivery and positioning system having a stabilizing guide wire 10 will now be described, as representative of its use in general for any type of interventional procedure, including percutaneous catheter procedure with reference to FIGS. 4-8 and for an exemplary heart procedure with reference to FIG. 14 compared to the use of a standard guide wire in the same heart procedure with reference to FIG. 13.

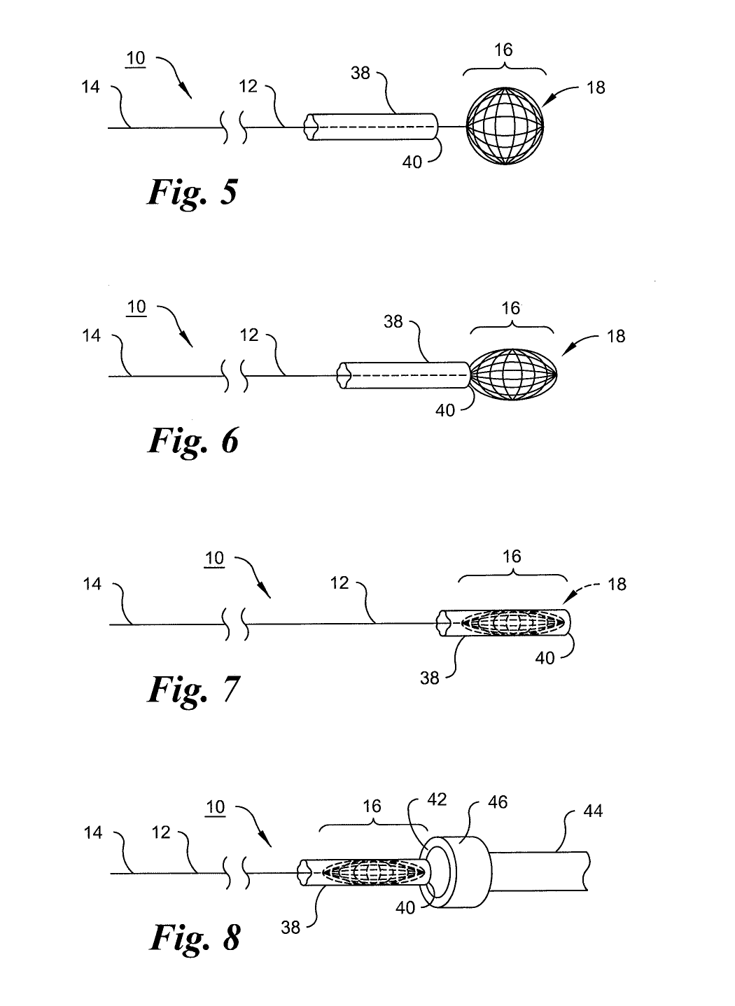

FIG. 4 shows the expandable portion 18 at the distal end 16 of the exemplary embodiment of the guide wire 10 of FIG. 3 in an expanded condition and a transfer tube 38, truncated at its proximal portion, inserted over the proximal end of the elongated member 12 of the guide wire 10. FIG. 5 shows the transfer tube 38, truncated at its proximal portion advanced along the elongated member 12 of the guide wire 10 toward the distal end 16 of the guide wire 10. Holding the transfer tube 38 firmly, the proximal end 14 of the guide wire 10 is pulled so that the expandable portion 18 is drawn into the transfer tube. FIG. 6 depicts the expandable portion 18 at the distal end 16 of the guide wire 10 beginning to be compressed into its compressed condition as it enters the distal end 40 of the transfer tube 38. FIG. 7 shows the expandable portion 18 at the distal end 16 of the guide wire 10 compressed into its compressed condition within the distal end 40 of the transfer tube 38, as the guide wire 10 is continued to be pulled and the expandable portion 18 is drawn fully into the transfer tube 38.

The transfer tube 38, with the guide wire 10, including the compressed expandable portion 18 inside the transfer tube, is then used as a guide to advance the compressed expandable portion 18 and guide wire 10 into the proximal end 42 of a delivery tube 44, which may be a standard hypodermic tube, typically but without limitation made of stainless steel as discussed above, as shown in FIG. 8. The delivery tube 44 may be any standard or specialized delivery tube or catheter for a diagnostic procedure in an animal or human, such as an, angiographic catheter. Other non-limiting examples of transfer and delivery devices include exchange catheters, sheaths or a tube within a tube type of coaxial guide wire system. The delivery tube 44 typically has at its proximal end 42 a standard Leur lock 46, shown schematically in FIG. 8, used for making leak-free connections between a male fitting and its mating female part on medical and laboratory instruments. Alternatively, any other type of connector providing a leak-free connection could be used.

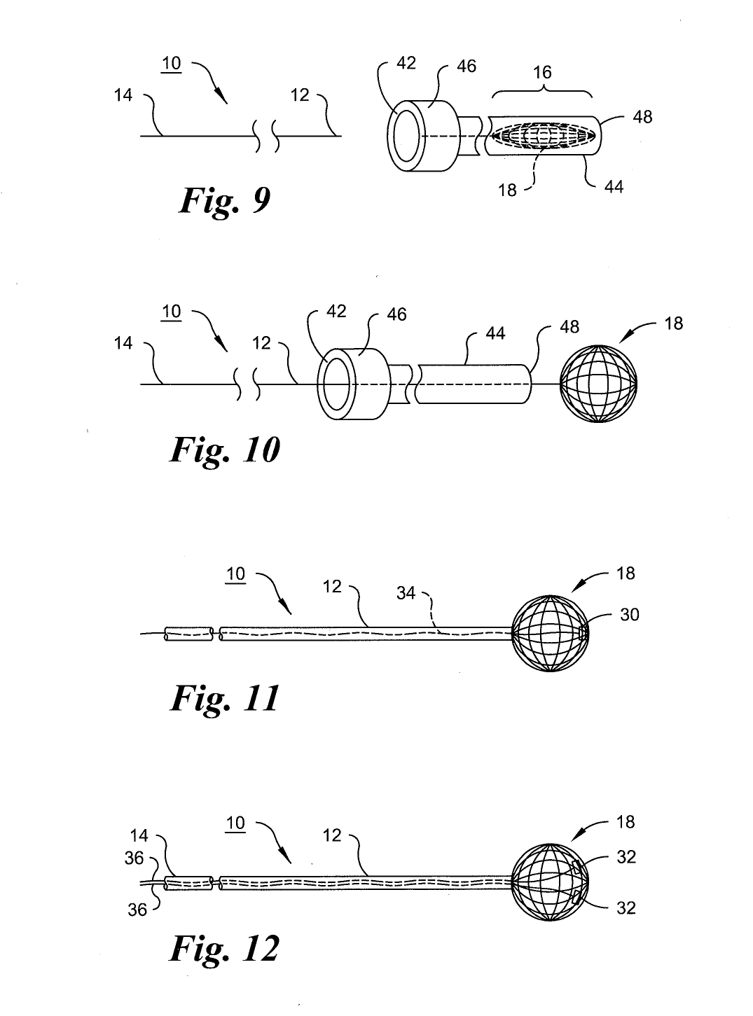

The transfer tube 38 and its contained guide wire 10 with the expandable portion 18 in its compressed condition is then moved along the length within the delivery tube, which has been previously approximately located in the desired location, within the targeted vessel, tissue or organ, for example the left ventricle of a heart, the delivery tube, having been approximately located using a standard guide wire that has already been withdrawn from the subject's body following the approximate placement of the delivery tube in the desired location. FIG. 9 shows the stabilizing guide wire 10, with the expandable portion 18 compressed within the delivery tube 44 after the transfer tube 38 has been removed from the delivery tube 44 and the guide wire 10, where the compressed expandable portion 18 advanced toward the distal end 48 of the delivery tube. An intermediate portion of the delivery tube 44 is shown as truncated for ease of illustration. FIG. 10 shows the expandable portion 18 at the distal end 16 of the guide wire 10 expanded into its expanded condition upon exiting from the distal end 48 of the delivery tube 44. At this position, the guide wire 10 can be manipulated and maneuvered into the final appropriate position within the targeted cavity of the tissue or organ for diagnostic or interventional procedures, such as in the apex of the left ventricle of a human heart.

Once the guide wire 10 is in place, the delivery tube 44 can be retracted from the body. The procedure of interest can be performed over the stabilizing guide wire 10 using another diagnostic or interventional catheter and its associated equipment, replacement valves or the like. At the end of the procedure, the diagnostic or interventional catheter may be removed from the body and another delivery tube 44 can be reintroduced over the guide wire 10, advanced toward the distal end 16 of the guide wire 10, and the expandable portion 18 can be retracted and compressed into its compressed condition within the distal end 48 of the delivery tube in the same manner used for inserting and compressing the expandable portion 18 into the transfer tube 38 as described above. Then the delivery tube and the guide wire 10 with the compressed expandable portion 18 can be removed as a single, combined unit. Alternatively, if the diagnostic or interventional catheter used in the procedure has sufficient structural integrity and a sufficiently large lumen to receive and compress the expandable portion 18 of the guide wire, and typically they do, the diagnostic or interventional catheter need not be removed and replaced with another delivery tube 44 to extract the guide wire 10 and its expandable portion 18. Instead, the guide wire 10 and its expandable portion 18 can be withdrawn and compressed into the diagnostic or interventional catheter and the guide wire 10 with its compressed expandable portion 18, together with the diagnostic or interventional catheter can be removed from the body as a single, combined unit.

Another aspect of the invention relates to methods of using the stabilizing guide wire 10 of the present invention in various percutaneous procedures involving a human or animal subject. Accordingly, this aspect relates to a method of performing a percutaneous procedure comprising inserting the guide wire 10 into a transfer tube 38, guiding the transfer tube 38 containing the guide wire 10 including its expandable portion 18 in a compressed condition through a delivery tube 44 into an approximate position within a vessel or cavity of a tissue or organ of a subject, retracting the transfer tube 38, extending the guide wire 10 from the delivery tube 44 such that the expanded portion 18 of the guide wire 10 expands to its expanded condition, and positioning the expanded portion 18 of the guide wire 10 in its expanded position to a final desired location within the vessel or cavity of a tissue or organ of the subject. Particularly preferred are methods involving surgical procedures within the human heart. Exemplary heart procedures include, without limitation, transcatheter aortic valve implementation (TAVI), transcatheter pulmonic valve implementation (TPVI), transcatheter tricuspid valve implementation, percutaneous mitral valve implementation, percutaneous mitral valve repair, transcatheter tricuspid valve repair, and mitral pulmonic and aortic valvuloplasty, among other procedures.

The use of the guide wire 10 will now be generally described for a typical TAVI procedure, with reference to FIG. 13, showing the use of a prior art guide wire for comparison with FIG. 14, showing the use of the guide wire 10 of the present invention, both figures showing the procedure with reference to a schematic representation of a living human heart 50, with some vessels and other heart structures removed for the sake of clarity. In both FIGS. 13 and 14, A designates the aorta, AV designates the aortic valve, IVC designates the inferior vena cava, LA designates the left atrium, LV designates the left ventricle, LVA designates the left ventricle apex, MV designates the mitral valve, RA designates the right atrium, RV designates the right ventricle, SVC designates the superior vena cava, and TV designates the tricuspid valve.

In the procedures using both the prior art guide wire 10′ of FIG. 13 and the guide wire 10 of the present invention, a delivery tube 44 is placed in approximate position in the left ventricle using standard procedures and a standard guide wire. Typically, for a TAVI procedure, an incision is made in the groin and a standard guide wire is advanced through the femoral artery to the aorta A, using fluoroscopy or other imaging technique to position the standard guide wire just past the aortic valve AV into the left ventricle LV. Then in the usual procedure depicted in FIG. 13, the standard prior art guide wire 10′ having an elongated portion 12′ and ending with a “J”-curve 52′ at its distal end 16′ is pushed further out of the distal end 48 of the delivery tube 44 until the J-curve 52′ is in the left ventricle apex LVA. Because the J-curve 52′ is acts essentially as a two-dimensional structure, it is difficult to place precisely within the left ventricle apex LVA and there is a risk of abrasion, puncture or other adverse consequences as the guide wire 10′ is manipulated and maneuvered into its final position. Accurate placement of the interventional catheter over the guide wire 10′ is difficult, after the delivery tube 44 is retracted, as the wire is not moveable in a predictable way and is not able to pivot. Because of the asymmetric J-curve 52′, when the proximal end of the guide wire 10′ is twisted, the elongated member 12′ of the guide wire 10′, particularly in an area designated as 12′a, close to the distal end 16′ of the guide wire, often comes into contact with the wall of the septum S separating the left ventricle LV from the right ventricle RV. This contact adversely affects the ability of the surgeon or other health care provider in precisely and finally locating the distal end 16′ of the guide wire 10′ in the desired position. When the guide wire 10′ is finally in place after considerable difficulty, the delivery tube 44 is retracted from the body and the interventional catheter, with the replacement aortic valve and ancillary equipment is guided over the guide wire 10′ to the appropriate location for the TAVI procedure. When the procedure is completed, the guide wire 10′ is retracted so that the J-curve 52′ is inside the catheter and the guide wire 10′ within the catheter and the catheter are removed from the subject's body as a single, combined unit.

In the TAVI procedure using the guide wire 10 of the present invention as schematically shown in FIG. 14, the guide wire 10 with its compressed expanded portion 18 is inserted via the transfer tube 38 into the pre-placed delivery tube 44 adjacent the distal end 48 of the delivery tube 44 that was approximately located in the left ventricle LV just past the aortic valve AV. Then the transfer tube 38 is withdrawn. The guide wire 10 is then extended from the distal end 48 of the delivery tube 44 where the expandable portion 18 expands to its expanded condition. Since the expanded portion 18 in its expanded position is in fact and acts as a three-dimensional structure that is conformable to the final desired location of the vessel, tissue or organ cavity, such as the left ventricle apex LVA, placement is precise. The expanded expandable portion 18 allows relative freedom of pivoting and other degrees of manipulation and maneuvering during final placement.

Because of the three-dimensional structure of the expanded expandable portion 18, such as in a generally ovoidal or generally spherical shape, the elongated member 12 of the guide wire 10 is generally centrally located and tends not to contact the side wall of the septum S within the left ventricle LV. This allows greater degrees of freedom for pivoting or other maneuvering the guide wire to the precise, final desired location in the left ventricle apex LVA. Also, since the expanded expandable portion 18 is blunt, there is less likelihood of abrasion, puncturing or other trauma associated with the placement or removal of the a guide wire 10 in its final position.

Once the guide wire 10 is in its final placement, the delivery tube 44 is removed from the subject's body. Based on the three-dimensional shape of the expanded expandable portion 18 firmly placed within the left ventricle apex LVA, there is less likelihood of displacement during removal of the delivery tube 44 or the placement of the interventional catheter with its replacement valve and ancillary items as may happen more readily with the J-curve 52′ at the distal end 16′ of the prior art guide wire 10′. As a result, the interventional catheter may be located more precisely for the very intricate TAVI procedure.

Once the procedure is completed, the guide wire 10 may be retracted into the interventional catheter to compress the expandable portion 18 into its compressed condition, and the guide wire 10 inside the catheter can be removed from the subject's body as a single, combined unit. If desired, when the TAVI procedure is completed, the interventional catheter can be removed first, and another delivery tube 44 can be positioned over the guide wire 10 in the left ventricle LV beyond the replace aortic valve AV. Then the guide wire 10 may be retracted into the delivery tube 44 to compress the expandable portion 18 into its compressed condition, and the guide wire 10 inside the delivery tube 44 can be removed from the subject's body as a single, combined unit.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as set forth above or as defined by the appended claims.

I claim:

1. An anatomic device delivery and positioning system having a stabilizing guide wire guiding placement of a delivery device into a vessel or cavity of a tissue or organ within a body of a living subject into which the guide wire is insertable, wherein the guide wire comprises an elongated member having a proximal end and a distal end, the proximal end to extend out of the body and the distal end to extend into the body, the distal end having an expandable portion that expands from a compressed condition when inside of a delivery tube or catheter to an expanded condition when outside of the delivery tube or catheter.

2. The system according to claim 1, wherein the elongated member comprises a metal wire.

3. The system according to claim 2, wherein, wherein the wire is at least partially coated with a synthetic polymer.

4. The system according to claim 3, wherein the synthetic polymer is selected from the group consisting of silicone, polytetrafluoroethylene and polyurethane.

5. The system according to claim 2, wherein the metal wire comprises a metal selected from the group consisting of titanium, vanadium, aluminum, nickel, iron, tantalum, zirconium, chromium, silver, gold, silicon, magnesium, niobium, scandium, platinum, cobalt, palladium, manganese, molybdenum, and alloys of any two or more thereof.

6. The system according to claim 5, wherein the metal wire comprises a metal selected from the group consisting of nitinol and stainless steel.

7. The system according to claim 1, wherein the metal wire has a diameter of about 0.009 inch (0.23 mm) to about 0.064 inch (1.63 mm).

8. The system according to claim 7, wherein the metal wire has a diameter of about 0.013 inch (0.33 mm) to about 0.050 inch (1.27 mm).

9. The system according to claim 8, wherein the metal wire has a diameter of about 0.035 inch (0.89 mm).

10. The system according to claim 1, wherein the elongated member has a flexural modulus of about 8 gigapascals [GPa] (about 8,000 newtons/square millimeter [N/mm²] or about 1,160,302 pounds per square inch [psi]) to about 200 GPa (about 200,000 N/mm² or about 29,007,548 psi).

11. The system according to claim 1, wherein the elongated member is an elongated tubular member.

12. The system according to claim 11, further comprising at least one pacing electrode at the expandable portion capable of contacting the tissue of the tissue or organ cavity into which it is insertable and having at least one lead wire extending through the elongated tubular member connected to the at least one electrode.

13. The system according to claim 12, wherein the at least one pacing electrode is a unipolar electrode or a bipolar electrode.

14. The system according to claim 1, wherein the expandable portion comprises a material having a characteristic that is at least one of a superelastic material, a plastically deformable material and an elastic material.

15. The system according to claim 14, wherein the material of the expandable portion comprises a metal selected from the group consisting of titanium, vanadium, aluminum, nickel, iron, tantalum, zirconium, chromium, silver, gold, silicon, magnesium, niobium, scandium, platinum, cobalt, palladium, manganese, molybdenum, and alloys of any two or more thereof.

16. The system according to claim 14, wherein the material of the expandable portion comprises a metal selected from the group consisting of nitinol and stainless steel.

17. The system according to claim 14, wherein the material of the expandable portion comprises nitinol.

18. The system according to claim 1, wherein the expandable portion when in the expanded condition has a generally spherical shape.

19. The system according to claim 18, wherein the expandable portion when in the expanded condition has a diameter of about 0.02 inch (0.5 mm) to about 1.57 inches (4 cm).

20. The system according to claim 19, wherein the expandable portion when in the expanded condition has a diameter of about 0.59 inch (1.5 cm) to about 1.18 inches (3 cm).

21. The system according to claim 20, wherein the expandable portion when in the expanded condition has a diameter of about 0.78 inch (2 cm).

22. The system according to claim 1, wherein the expandable portion when in the expanded condition has the ability to conform to the shape of a portion of the vessel or tissue cavity into which it is to be inserted without adversely affecting the vessel or tissue cavity.

23. The system according to claim 1, wherein the expandable portion when in the expanded condition has the ability to self-center within the vessel or tissue cavity into which it is to be inserted.

24. The system according to claim 1, wherein the expandable portion when in the expanded condition has a generally spherical shape.

25. The system according to claim 1, wherein the expandable portion when in the expanded condition has a generally ovoidal shape.

26. The system according to claim 1, wherein the tissue cavity is a chamber of a heart.

27. The system according to claim 26, wherein the heart is a human heart.

28. The system according to claim 1, wherein the expandable portion when in the compressed condition has a diameter of about 0.01 inch (0.25 mm) to about 0.082 inch (2.3 mm).

29. The system according to claim 28, wherein the expandable portion when in the compressed condition has a diameter of about 0.056 inch (1.42 mm) to about 0.070 inch (1.78 mm).

30. A method of performing a percutaneous procedure within a vessel or cavity of a tissue or organ of a subject comprising
inserting the guide wire of claim 1 into a transfer tube,
guiding the transfer tube containing the guide wire including its expandable portion in a compressed condition through a delivery tube into an approximate position within a vessel or cavity of a tissue or organ of a subject,
retracting the transfer tube,
extending the guide wire from the delivery tube such that the expanded portion of the guide wire expands to its expanded condition, and
positioning the expanded portion of the guide wire in its expanded position to a final desired location within the vessel or cavity of the tissue or organ of the subject.

31. The method of claim 30 wherein the procedure is performing a transcatheter heart procedure comprising inserting a catheter onto the guide wire inserted into a cavity in the heart.

32. The method of claim 31, wherein the heart is a human heart.

33. The method of claim 32, wherein the cavity within the heart is the left ventricle accessed via the aorta.

34. The method according to claim 31, wherein the procedure is a transcatheter aortic valve implementation procedure.

35. The method according to claim 31, wherein the procedure is a transcatheter pulmonic valve implementation.

36. The method according to claim 31, wherein the procedure comprises percutaneous mitral valve repair.

37. The method according to claim 31, wherein the procedure comprises percutaneous mitral valve implementation.

38. The method according to claim 31, wherein the procedure comprises transcatheter tricuspid valve repair.

39. The method according to claim 31, wherein the procedure comprises mitral valvuloplasty.

40. The method according to claim 31, wherein the procedure comprises transcatheter tricuspid valve implementation.

41. The method according to claim 31, wherein the procedure comprises aortic valvuloplasty.

42. The method according to claim 31, wherein the procedure comprises pulmonic valvuloplasty.
